(19) United States
(12) Patent Application Publication
(10) Pub. No.: US 2002/0127548 A1
(43) Pub. Date: Sep. 12, 2002

(54) METHODS FOR DETECTING MUTATIONS ASSOCIATED WITH HYPERTROPHIC CARDIOMYOPATHY

(76) Inventors: CHRISTINE SIEDMAN, MILTON, MA (US); JONATHAN SEIDMAN, MILTON, MA (US); LUDWIG THIERFELDER, BERLIN (DE); HUGH WATKINS, HEADINGTON (GB); CALUM MCRAE, BROOKLINE, MA (US)

Correspondence Address: LAHIVE & COCKFIELD, LLP, 28 STATE STREET, BOSTON, MA, 02109

(21) Appl. No.: 08/647,444

(22) Filed: Nov. 30, 1995

Related U.S. Application Data
(63) Continuation-in-part of application No. 08/354,326, filed on Dec. 12, 1994, now Pat. No. 5,912,121.
Application No. 08/354,326 is a continuation of application No. 08/252,627, filed on Jun. 2, 1994, now abandoned.
Application No. 08/252,627 is a continuation-in-part of application No. 07/989,160, filed on Dec. 11, 1992, now Pat. No. 5,429,923.

(51) Int. Cl. C12Q 1/70; C12Q 1/68; C07H 21/02; C07H 21/04; C12P 19/34

(52) U.S. Cl. 435/6; 435/5; 435/91.2; 536/23.1; 536/24.3; 536/24.31; 536/24.33

(57) ABSTRACT

The invention pertains to methods for detecting the presence or absence of a mutation associated with hypertrophic cardiomyopathy (HC). The methods include providing DNA which encodes a cardiac myosin binding protein and detecting the presence or absence of a mutation in the amplified product which is associated with HC. The invention further pertains to methods for diagnosing HC in a subject. These methods typically include obtaining a sample of DNA which encodes a cardiac myosin binding protein from a subject being tested for FHC and diagnosing the subject for FHC by detecting the presence or absence of a mutation in the sarcomeric thin filament protein which causes FHC as an indication of the disease. Other aspects of the invention include kits useful for diagnosing HC and methods for treating HC.

### RELATED APPLICATIONS

This application is a continuation-in-part application of Ser. No. 08/354,326 filed on Dec. 12, 1994, now pending, which is a continuation of Ser. No. 08/252,627 filed on Jun. 2, 1994, which is a continuation-in-part application of Ser. No. 07/989,160, filed Dec. 11, 1992, now issued U.S. Pat. No. 5,429,923. The contents of all of the aforementioned applications and/or issued patent are expressly incorporated herein by reference.

### GOVERNMENT SUPPORT

This work was supported, in part, by grants from the National Institutes of Health

### BACKGROUND OF THE INVENTION

Familial hypertrophic cardiomyopathy (hereinafter FHC) is a primary and inherited disorder of heart muscle that is characterized by increased ventricular mass, hyperkinetic systolic function and impaired diastolic relaxation. Goodwin, J. F. et al. (1961) Br. Med J 21:69-79. The pathological features of this disorder are well established (Maron, B. J. and Epstein, S. E. (1980) Amer. J. Cardiol 45:141-154). In addition to the classical finding of asymmetrical thickening of the intraventricular septum, hypertrophy of the adjacent left ventricular anterior free wall, apex or right ventricle can also occur. Hence the anatomical distribution and severity of hypertrophy can vary considerably. Maron, B. J. et al. (1981) Amer. J. Cardiol. 48:418-428. Fibrosis occurs within the hypertrophied ventricle and a fibrotic plaque is frequently demonstrable over the septal region that apposes the anterior mitral valve leaflet during systole. Other gross pathological findings include atrial dilation and thickening of the mitral valve leaflets. Roberts, W. C. and Ferrans, V. J. (1975) Hum Pathol. 6:287-342.

The most characteristic histological abnormalities seen in FHC are in myocyte and myofibrillar disarray. Davies, M. J. (1984) Br. Heart. J. 51:331-336. Myocytes can be hypertrophied to ten to twenty times the diameter of a normal cardiac cell and may contain hyperchromatic, bizarre nuclei. Becker, A. E. (1989) Pathology of Cardiomyopathies in Cariomyopathies: Clinical Presentation, Differential Diagnosis, and Management (Shaver. J. A. ed.) F. A. Davis Co., New York, pp. 9-31. Cells are arranged in a disorganized fashion with abnormal bridging of adjacent muscle fibers and intercellular contacts, producing whorls. Ultrastructural organization is also distorted: myofibrils and myofilaments are disoriented with irregular Z bands. Ferrans, V. J. et al. (1972) Circulation 45:769-792. While the histopathological features overlap Keith those seen in hypertrophy that is secondary to other diseases, the extent of ventricular involvement and the severity of myocyte and myofibrillar disarray are considerably greater in FHC.

The pathology of FHC typically results in the physiological consequences of both systolic and diastolic dysfunction. Maron, B. J. et al. (1987) N. Eng. J. Med, 316:780-789. Systolic abnormalities include rapid ventricular emptying, a high ejection fraction and the development of a dynamic pressure gradient. Reduced left ventricular compliance results from an increase in the stiffness of the hypertrophied left ventricle and an increase in left ventricular mass. Impaired relaxation produces elevated diastolic pressures in the left ventricle as well as in the left atrium and pulmonary vasculature.

The clinical symptoms in individuals with FHC are variable and may reflect differences in the pathophysiological manifestations of this disease. Frank, S. and Braunwald, E. (1968) (Circulation 37:759-788 Affected individuals frequently present with exertional dypsnea, reflecting the diastolic dysfunction that characterizes this disease. Angina pectoris is a common symptom, despite the absence of coronary artery disease. Ischemia may result from increased myocardial demand as well as inappropriately reduced coronary flow due to increased left ventricular diastolic pressures. Sudden, unexpected death is the most serious consequence of FHC, and occurs in both asymptomatic and symptomatic individuals.

The diagnosis of FHC relies on the presence of typical clinical symptoms and the demonstration of unexplained ventricular hypertrophy. Maron, B. J. and Epstein, S. E. (1979) Amer. J Cardiol 43:1242-1244; McKenna, W J. et al. (1988) J. Amer Coll Cardinol. 11:351-538. Two-dimensional echocardiography and doppler ultrasonography are used to quantitate ventricular wall thickness and cavity dimensions, and to demonstrate the presence or absence of systolic anterior motion of the mitral valve. Electrocardiographic findings include bundle-branch block, abnormal Q waves and left ventricular hypertrophy with repolarization changes. Despite the existence of these detection tools, diagnosis of FHC can be difficult, particularly in the young, who may exhibit hypertrophy only after adolescent growth has been completed. Maron, B. J. et al. (1987). N Eng J Med 316:780-789.

Recently, genetic analyses have enabled identification of mutations in the β cardiac myosin heavy chain gene which are associated with FHC. Seidman, C. E. and Seidman, J G. (1991 ) Mol Biol Med 8:159-166. The β cardiac myosin heavy chain gene encodes a sarcomeric thick filament protein.

### SUMMARY OF THE INVENTION

The present invention is based, at least in part, on the discover of mutations in a gene encoding a cardiac myosin binding protein, e.g., cardiac myosin binding protein-C, which cause hypertrophic cardiomyopathy (hereinafter HC) result in HC.

The present invention provides methods for diagnosing individuals as having HC e g. familial or sporadic hypertrophic cardiomyopathy (hereinafter FHC or SHC). The methods provide a useful diagnostic tool which becomes particularly important when screening asymptomatic individuals suspected of having the disease. Symptomatic individuals have a much better chance of being diagnosed properly by a physician. Asymptomatic individuals from families having a history of FHC can be selectively screened using the method of this invention allowing for a diagnosis prior to the appearance of any symptoms. Individuals having the mutation responsible for FHC can be counseled to take steps which hopefully will prolong their life. i.e. avoiding rigorous exercise.

The invention pertains to methods for detecting the presence or absence of a mutation associated with HC. The methods include providing DNA which encodes a cardiac myosin binding protein and detecting the presence or absence of a mutation in the DNA which is associated with HC. The methods can include amplifying the DNA (e.g., using a polymerase chain reaction. e.g., a nested polymerase chain reaction) to form an amplified product and detecting the presence or absence of mutations in the amplified product which are associated with HC. In one embodiment of the invention, the mutation associated with HC is detected by contacting the DNA with an RNA probe completely hybridizable to DNA which encodes a normal cardiac myosin binding protein. The RNA probe and the DNA encoding a normal cardiac myosin binding protein form a hybrid double strand having an unhybridized portion of the RNA strand at any portion corresponding to a hypertrophic cardiomyopathy-associated mutation in the DNA strand. The presence or absence of an unhybridized portion of the RNA strand can then be detected as an indication of the presence or absence of a HC-associated mutation in the corresponding portion of the DNA strand. These methods can optionally include contacting the hybrid double strand with an agent capable of digesting an unhybridized portion of the RNA strand prior to the detecting step.

Examples of cardiac myosin binding protein DNA which can be analyzed using the methods of the invention include DNA which encodes cardiac myosin binding protein-C and cardiac myosin binding protein-II. The mutations in the DNA which encodes a cardiac myosin binding protein include point mutations (e.g., missense mutations), duplication mutations or splice site mutations. In one embodiment of the invention, the DNA which encodes a cardiac myosin binding protein is cDNA reverse transcribed from RNA. An example of a source of RNA to be used as a template for reverse transcription is nucleated blood cells (e.g., lymphocytes).

The invention still further pertains to methods for diagnosing FHC in a subject. The methods include obtaining a sample of DNA which encodes a cardiac myosin binding protein from a subject being tested for FHC and diagnosing the subject for FHC by detecting the presence or absence of a mutation in the cardiac myosin binding protein which causes hypertrophic cardiomyopathy as an indication of the disease. The method optionally includes amplifying the cardiac myosin binding protein DNA prior to the diagnosing step. In one embodiment of the invention, the cardiac myosin binding protein is cardiac myosin binding protein-C and the mutation is either a duplication mutation or splice site mutation

Other aspects of the invention includes methods for detecting the presence or absence of a mutation associated with HC (e.g., FHC or SHC) which include providing DNA which encodes a cardiac myosin binding protein and detecting the presence or absence of a mutation in the DNA which is associated with HC. The methods can include amplifying the DNA (e.g., using a polymerase chain reaction, e.g., a nested polymerase chain reaction) to form an amplified product and detecting the presence or absence of mutations in the amplified product which are associated with HC. In one embodiment of the invention, the cardiac myosin binding protein is cardiac myosin binding protein-C and the mutation is either a duplication mutation or splice site mutation.

Still other aspects of the invention include non-invasive methods for diagnosing HC. These methods typically include obtaining a blood sample from a subject being tested for HC (e.g., either FHC or SHC), isolating cardiac myosin binding protein RNA from the blood sample, and diagnosing the subject for HC by detecting the presence or absence of a mutation in the RNA which is associated with HC as an indication of the disease. In one embodiment of the invention, the presence or absence of a mutation associated with HC in the RNA is detected by preparing cardiac myosin binding protein cDNA from the RNA to form cardiac myosin binding DNA and detecting mutations in the DNA as being indicative of mutations in the RNA. The methods can optionally include amplifying the cardiac myosin binding protein DNA prior to detecting a mutation in the DNA which is associated with HC and/or evaluating the subject for clinical symptoms associated with HC.

Other aspects of the invention include kits useful for diagnosing HC. The kits typically contain a first container holding an RNA probe completely hybridizable to DNA which encodes a cardiac myosin binding protein (e.g., cardiac myosin binding protein-C). The kits can further optionally contain a second container holding primers useful for amplifying the DNA which encodes a cardiac myosin binding protein. The kits can also optionally contain a third container holding an agent for digesting unhybridized RNA and or instructions for using the components of the kits to detect the presence or absence of mutations in amplified DNA which encodes a cardiac myosin binding protein.

The invention further features a non-human embryo comprising DNA which encodes a cardiac myosin binding protein. The DNA contained in the nonhuman embryo has at least one hypertrophic cardiomyopathy-causing mutation in its nucleotide sequence.

The invention also features a non-human animal comprising DNA which encodes a cardiac myosin binding protein. The DNA contained in the non-human animal has at least one hypertrophic cardiomyopathy-causing mutation in its nucleotide sequence.

Other aspects of the invention include methods for screening an agent for its ability to treat hypertrophic cardiomyopathy in a subject. These methods include providing a non-human animal comprising DNA which encodes a cardiac myosin binding protein, the DNA having at least one hypertrophic cardiomyopathy-causing mutation in its nucleotide sequence, administering an agent being tested for its ability to treat hypertrophic cardiomyopathy in a subject to the non-human animal, and determining the effect of the agent on the hypertrophic cardiomyopathy in the non-human animal.

Further aspects of the invention include methods for treating hypertrophic cardiomyopathy in a subject. These methods include providing DNA which encodes a normal cardiac myosin binding protein and administering the DNA to a subject having hypertrophic cardiomyopathy such that the hypertrophic cardiomyopathy is treated.

### BRIEF DESCRIPTION OF THE DRAWINGS

FIG. 1 is a schematic depicting the pedigrees of Families NN and CD. Clinical affection status is indicated: darkened, affected; clear, unaffected; stippled, indeterminate (see text). Genetic affection status also is indicated: +, mutation present; −, mutation absent. Genetic studies were performed on all surviving first degree relatives except individual III-2 in Family NN.

FIG. 2 is a schematic depicting the cardiac MyBP-C gene structure in the region of exon M. Nucleotide residues defining exon-intron boundaries are numbered below: exons are denoted arbitrarily by letter. The G→C transversion at position 5 of the 5′ splice donor sequence (underlined) is indicated: the mutation creates a new BstEII site. The positions and orientation of primers are shown and approximate sizes of introns given.

FIG. 3 is a schematic depicting the cardiac MyBP-C gene structure in the region of the duplication The duplication occurs in the penultimate exon of the coding sequence denoted exon P; the termination codon (TGA) is indicated in exon Q. The 18 duplicated nucleotides, and the amino acid residues encoded are in bold.

FIG. 4 is a schematic showing normal and mutant MyBP-C polypeptides. a The normal structure of cardiac MyBP-C (based on M. Gautel et.al. (1995) EMBO J 14 1952-1960): almost the entire protein is taken up by the seven immunoglobulin-1, or immunoglobulin C2, repeats Ig-1) and three fibronectin type 3 repeats (fn-3) characteristic of other myosin binding proteins (K. T. Vaughan, et.al. (1992) Symp Soc. Exp Biol 46 167-177, F. E. Weber, et.al. (1993) Eur. J. Biochem 216:661-669). In addition a 103 bp sequence characteristic only of other MyBP-Cs is indicated as the MyBP-C motif (M Gautel et.al. (1995) EMBO J. 14: 1952-1960). The high affinity myosin heavy chain binding domain (confined to the C10 Ig-1 repeat (T. Okagaki, et.al. (1993) J. Cell Biol 123:619-626) is indicated. Amino acid residue numbers are according to (M. Gautel et al (1995) EMBO J 14: 1952-1960) (in which spaces have been introduced to maximize homology) b The predicted product of the aberrantly spliced MyBP-C cCDNA in Family NN. Skipping of the 140 bp exon M results in loss of the terminal 213 amino acid residues—including the C10 Ig-1 repeat; a frameshift encodes 37 novel residues followed by premature termination. c. The predicted product of the MyBP-C cDNA with the tandem duplication in Family CD: the region occupied by six duplicated amino-acid residues is indicated.

FIGS. 5A-J contain sequence information for primers used in the Example below.

### DETAILED DESCRIPTION OF THE INVENTION

The invention provides a method for detecting the presence or absence of a mutation associated with HC which comprises providing DNA which encodes a cardiac myosin binding protein and detecting the presence or absence of a mutation in the DNA which is associated with HC. The methods can further comprise amplifying the DNA (e.g., using a polymerase chain reaction, e.g., a nested polymerase chain reaction) to form an amplified product and detecting the presence or absence of mutations in the amplified product which are associated with HC.

For purposes of this invention, the term “mutation” is intended to include mutations associated with the respective diseases being discussed, e.g. HC. The mutation can be a gross alteration in the RNA or DNA or a small alteration in the RNA or DNA (e.g. a point mutation in the RNA or DNA). Examples of common mutations are deletions and insertions of nucleotides. The mutation further can be a mutation of the DNA which changes the amino acid encoded by that portion of the DNA strand, e.g. a missense mutation, or a mutation which does not change the encoded amino acid. The term mutation also specifically includes splice site mutations (e.g., 5′ splice site donor mutations) or duplication mutations. Examples of specific mutations in the cardiac myosin binding protein-C gene which cause HC are described in the example below.

HC is a well characterized disorder or disease which is described in details in the Background of the Invention section. This term is intended to include FHC, SHC and secondary cardiac hypertrophy. Mutations resulting in FHC are inherited throughout families and mutations resulting in SHC occur sporadically without a traceable hereditary path. For example, a subject having HC clinical symptoms may be diagnosed as having SHC if both of the subject's parents are actually diagnosed and determined to be healthy yet the subject has HC. Even further, if an afflicted subject's parents are not available for diagnosis and the afflicted subject has no other known family members with HC, then the subject probably would be diagnosed as having SHC. Secondary cardiac hypertrophy occurs in response to different stimuli (e.g., hypertension) and shares morphologic and histologic features with FHC.

The term “amplification” for purposes of this invention is intended to include any method or technique capable of increasing in number the respective DNA (including culturing) or RNA being discussed. The preferred amplification techniques is the polymerase chain reaction (PCR) which is an art recognized technique and most preferably the amplification is conducted using a nested PCR technique as described in the examples below.

The phrase “DNA which encodes a cardiac myosin binding protein” for purposes of this invention includes both generic DNA which encodes a cardiac myosin binding protein and cDNA which encodes a cardiac myosin binding protein. The preferred DNA which encodes a cardiac myosin binding protein is cDNA reverse transcribed from RNA obtained from a subject being screened for the respective disorder or disease, e.g. SHC or FHC. The RNA may be obtained from cardiac or skeletal tissue or from nucleated blood cells as described below.

The detection of the presence or absence of a mutation associated with HC in an amplified product can be conducted using any method capable of detecting such mutations. Examples of conventional methods used to detect mutations in DNA sequences include direct sequencing methods (Maxim and Gilbert, (1977) Proc. Natl. Acad. Sci USA 74:560-564; Sanger et al. (1977) Proc. Natl. Acad Sci USA 74:5463-5467 (1977)), homoduplex methods, heteroduplex methods, the single-stranded confirmation of polymorphisms (SSCP analysis) technique, and chemical methods. It should be understood that these methods are being provided merely to illustrate useful methods and one of ordinary skill in the art would appreciate other methods which would be useful in the present invention. The preferred detection method of the present invention is a heteroduplex method, particularly a protection assay which is similar to the RNase protection assay described by Myers et al. ((1985) Science, 230(3):1242-46), the contents of which are expressly incorporated herein by reference.

A protection assay can be used to detect the presence or absence of the HC—causing mutation by combining amplified cardiac myosin binding protein DNA with an RNA probe under hybridization conditions forming a hybrid double strand. The RNA probe is selected to be completely hybridizable to DNA which encodes a normal cardiac myosin binding protein, i.e. DNA without disease-causing mutations. The hybridization conditions are the same or similar to those described by Myers et al., supra. For example, the hybridization can include the addition of the RNA probe to a solution containing the DNA, e.g. a hybridization buffer, at appropriate conditions, e.g. 90° C. for ten minutes. Subsequently, this mixture may be incubated for a longer period of time, e.g. at 45° C. for thirty minutes.

The term “completely hybridizable” for purposes of this invention is intended to include RNA probes capable of hybridizing at each nucleotide of a complementary normal DNA sequence. This characteristic of the RNA probe allows for the detection of an unhybridized portion at a mismatched or mutant nucleotide(s).

The hybrid double strand, i.e. the RNA:DNA double strand, has unhybridized portions of RNA at locations or portions corresponding to a mutation in the normal DNA strand, e g. an HC-associated mutation. The hybrid double strand can be contacted with an agent capable of digesting an unhybridized portion(s) of the RNA strand, e.g an RNase The presence or absence of any unhybridized portions are then detected by analyzing the resulting RNA products The RNA products can be analyzed by electrophoresis in a denaturing gel Two new RNA fragments will be detected if the sample DNA contained a point mutation resulting in an unhybridized portion recognizable by the RNase. The total size of these fragments should equal the size of the single RNA fragment resulting from the normal DNA. The mutation(s) can be localized relative to the ends of the RNA probe by determining the size of the new RNA products. The sequence of the mutation may be determined by looking at the localized portion of corresponding DNA.

The agent capable of digesting an unhybridized portion of the RNA strand can be any agent capable of digesting unprotected ribonucleotides in the hybrid strands. Examples of such agents include ribonucleases, particularly RNase A.

As set forth above, the method of this invention can detect the presence or absence of the mutation associated with the respective disease or even further, the position within the gene or sequence of the mutation. The sequence or position can be determined by observing fragments resulting from mutations and comparing the fragments to a known template derived from the riboprobe which is representative of normal DNA.

The present invention also pertains to methods for diagnosing familial hypertrophic cardiomyopathy in a subject. These methods include obtaining a sample of DNA which encodes a cardiac myosin binding protein from a subject being tested for familial hypertrophic cardiomyopathy and diagnosing the subject for familial hypertrophic cardiomyopathy by detecting the presence or absence of a mutation in the cardiac myosin binding protein which causes hypertrophic cardiomyopathy as an indication of the disease. These methods can include an additional step of amplifying the cardica myosin binding protein DNA prior to the diagnosing step. Exons suspected of containing the HC-causing mutation can be selectively amplified.

The term “subject” for purposes of this invention is intended to include subjects capable of being afflicted with HC. The preferred subjects are humans.

Other aspects of the present invention are non-invasive methods for diagnosing hypertrophic cardiomyopathy. The method involves obtaining a blood sample from a subject being tested for HC, isolating cardiac myosin binding RNA from the blood sample, and diagnosing the subject for HC by detecting the presence or absence of a HC-associated mutation in the RNA as an indication of the disease. In one embodiment of the invention, the presence or absence of a mutation associated with HC in the RNA is detected by preparing cardiac myosin binding protein cDNA from the RNA to form sarcomeric thin filament DNA and detecting mutations in the DNA as being indicative of mutations in the RNA. In this embodiment, the cardiac myosin binding protein DNA can be amplified prior to detecting a mutation in the DNA which is associated with HC. The subject can be further evaluated for clinical symptoms associated with HC (some of which are described in detail in the Background of the Invention section)

The RNA can be isolated from nucleated blood cells. Nucleated blood cells include lymphocytes, e.g. T and B cells, monocytes, and polymorphonuclear leukocytes. The RNA can be isolated using conventional techniques such as isolation from tissue culture cells, guantidinium methods and the phenol/SDS method. See Ausebel et al. (Current Protocols in Molecular Biology (1991), Chapter 4, Sections 4.1-4.3), the contents of which are expressly incorporated by reference.

The present invention is partly based on the discovery that normal and mutant cardiac myosin binding protein RNA is present in nucleated blood cells, e.g. lymphocytes, a phenomenon called ectopic transcription. Access to RNA provides a more efficient method of screening for disease-causing mutations because intron sequences have been excised from these transcripts. The present invention is a non-invasive method in that the mRNA is easily obtained from a blood sample.

The present invention also pertains to kits useful for diagnosing HC. The kits contain a first container such as a vial holding an RNA probe. The kits can further optionally contain a second container holding primers. The RNA probe is completely hybridizable to DNA which encodes a cardiac myosin binding protein and the primers are useful for amplifying DNA which encodes a sarcomeric thin filament protein. The kits can further contain an RNA digesting agent and/or instructions for using the components of the kits to detect the presence or absence of HC-associated point mutation in amplified DNA encoding a cardiac myosin binding protein.

Other aspects of the invention include non-human animal embryos comprising DNA which encodes a cardiac myosin binding protein. The DNA which encodes a cardiac myosin binding protein has at least one hypertrophic cardiomyopathy-causing mutation in its nucleotide sequence.

The term “non-human animal embryo” is intended to include a non-human fertilized embryo comprising at least one cell. Typically, a nonhuman embryo is derived from an animal of the class Mammalia. Examples of non-human mammals include dogs, cats, horses, cows, goats, rats, and mice.

The DNA can be introduced into the non-human embryo using any of the methods known in the art. Examples of well known methods of inserting DNA into a cell include calcium phosphate-mediated DNA transfection, electroporation, microinjection of the DNA into a non-human embryo, and virus-mediated delivery of the DNA to the embryo e.g. using retroviral vectors or adenovirus-based vectors.

The invention also pertains to non-human animals comprising DNA which encodes a cardiac myosin binding protein, the DNA having at least one hypertrophic cardiomyopathy-causing mutation in its nucleotide sequence. The term “non-human animal” is intended to include an animal that is not a human. Typically, the non-human animal is a mammal such as a mouse or rat.

Still other aspects of the invention include methods for screening agents for their ability to treat hypertrophic cardiomyopathy in a subject. These methods include providing a non-human animal comprising DNA which encodes a cardiac myosin binding protein, the DNA having at least one hypertrophic cardiomyopathy-causing mutation in its nucleotide sequence, administering an agent being tested for its ability to treat hypertrophic cardiomyopathy in a subject to a the non-human animal, and determining the effect of the agent on the hypertrophic cardiomyopathy in the nonhuman animal.

The agent being tested for its ability to treat hypertrophic cardiomyopathy can be administered to a subject at a level which is not detrimental to the subject. Examples of routes of administration which can be used include injection (subcutaneous, intravenous, parenteral, intraperitoneal, etc.), enteral, transdermal, and rectal.

The phrase “an agent being tested for its ability to treat hypertrophic cardiomyopathy” is intended to include a compound which can be tested to determine its ability to reduce, eliminate, or prevent the detrimental effects of HC on a subject.

The phrase “determining the effect of the agent on the HC in the non-human animal” is intended to include ascertaining whether the agent reduces, eliminates, or prevents the detrimental effects of HC on a subject or whether the agent has no effect on the detrimental effects of HC on a subject.

The term “treat” as used herein is intended to include reduction, elimination, or prevention of the detrimental effects (e.g., symptoms) of HC on a subject. Many of these detrimental effects are described in detail in the Background of the Invention section.

The invention further pertains to methods for treating hypertrophic cardiomyopathy in a subject comprising administering DNA which encodes a normal cardiac myosin binding protein to a subject having hypertrophic cardiomyopathy such that the hypertrophic cardiomyopathy is treated. These methods typically include packaging the DNA in a carrier such as a plasmid, phase (e.g., bacteria phage lambda), virus, or a lipid vesicle for enabling introduction of the DNA into a cell of the subject. Examples of viruses that are commonly used to deliver DNA to a target cell include retroviruses and vaccinia viruses. Preferred DNA carriers include viruses such as adenovirus and adeno-associated viruses. Examples of lipid vesicles include detergent or other amphipathic molecule micelles, membrane vesicles, liposomes, virosomes, and microsomes.

Lipid vesicles can also be used to deliver a normal cardiac myosin binding protein to a cell of a subject having hypertrophic cardiomyopathy such that the hypertrophic cardiomyopathy is treated.

The term “normal” as used herein is intended to refer to a protein which preforms its intended function. Normal proteins do not contain mutations which detrimentally effect the intended function of the protein.

The present invention is further illustrated by the following Example which in no way should be construed as further limiting. The entire contents of all of the references (including literature references, issued patents, published patent applications, and co-pending patent applications) cited throughout this application are hereby expressly incorporated by reference The entire contents of Rozensweig, A. et al. (1991) N. Eng. J Med 325:1753-60 (Dec. 19, 1991)) and Watkins, H. et al. (1992) N. Eng. J. Med. 326:1108-1114 also are expressly incorporated by reference.

### THE FOLLOWING MATERIALS AND METHODS APPLY TO THE EXAMPLES

Family Studies

Clinical evaluations, electrocardiographic and echocardiographic studies were performed as previously described (Watkins, H. et.al. (1995) N. Engl. J. Med. 332: 1058-1064). At the time of clinical evaluation, a blood sample was obtained for genetic analyses. Clinically unaffected individuals under the age of 16 years were excluded from linkage analyses. One clinically affected individual of Family NN (III-2) declined genetic testing. All studies were carried out in accordance with the guidelines of the Brigham and Women's Hospital Human Subjects Committee and the Abbott Northwestern Hospital Institutional Human Research Committee.

Cardiac MyBP-C Oligonucleotides

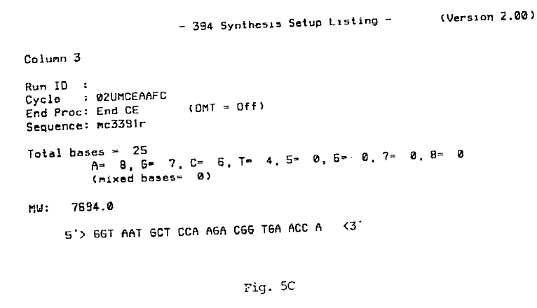

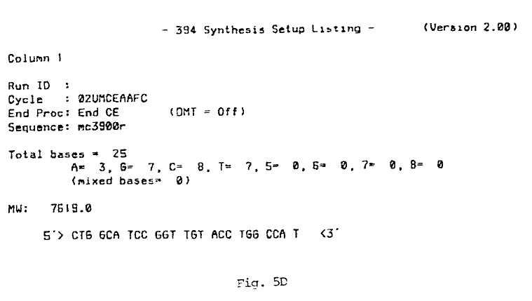

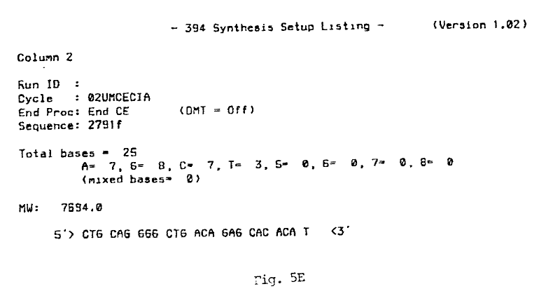

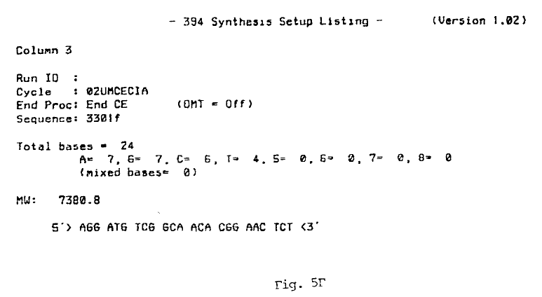

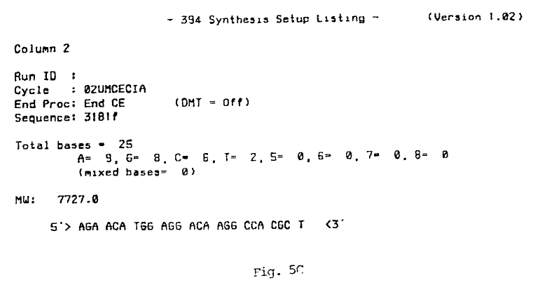

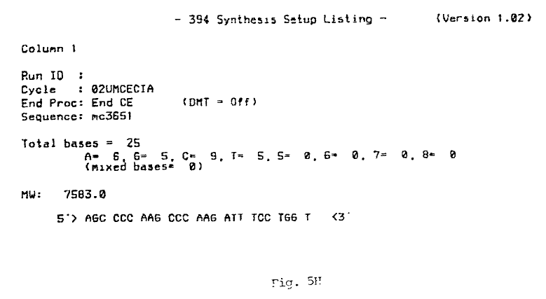

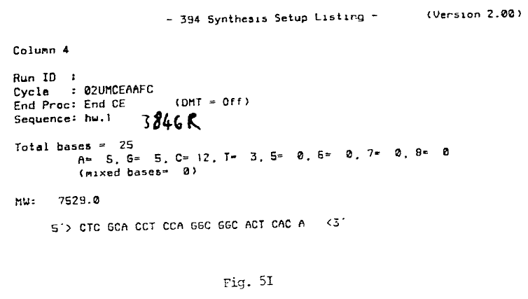

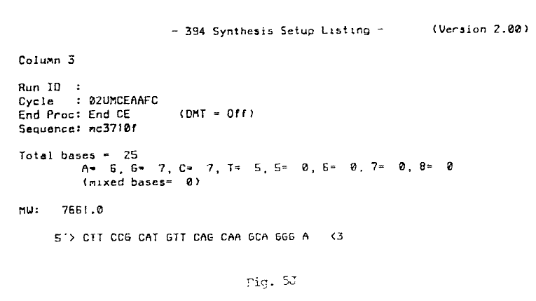

All oligonucleotides were 25mer synthesized according to the published cDNA sequence and numbered according to the position of the 5′ residue (Gautel, M. et.al. (1995) EMBO J. 14:: 1952-1960). F indicates forward, and R reverse, orientation. The sequence of all oligonucleotides discussed herein are provided below and in FIGS. 5A-5J:

Sequence No.

| | |
|---|---|
| 2761 | 5′ > CAG AGG GCT GCT CAG AGT GGG TGG < 3′ |
| 3930 | 5′ > CAA CTT CCC TCC AGG CTC CTG GCA C < 3′ |
| 3391 | 5′ > GGT AAT GCT CCA AGA CGG TGA ACC A < 3′ |
| 3900 | 5′ > CTG GCA TCC GGT TGT ACC TGG CCA T < 3 |
| 2791 | 5′ > CTG CAG GGG CTG ACA GAG CAC ACA T < 3′ |
| 3301 | 5′ > AGG ATG TCG GCA ACA CGG AAC TCT < 3′ |
| 3181 | 5′ > AGA ACA TGG AGG ACA AGG CCA CGC T < 3′ |
| 3651 | 5′ > AGC CCC AAG CCC AAG ATT TCC TGG T < 3′ |
| 3846 | 5′ > CTC GCA CCT CCA GGC GGC ACT CAC A < 3′ |
| 3701 | 5′ > CTT CCG CAT GTT CAG CAA GCA GGG A < 3′ |

Identification of a YAC Clone Carrying Cardiac MyBP-C

Human YAC DNA pools from the CEPH B library were searched by PCR according to instructions (library pools can be obtained from Research Genetics, Inc.). Primers 3301F and 3391R were chosen because they were found to span an intron, giving a single 315 bp product from genomic DNA (FIG. 2).

Amplification of cDNA Sequences

Using previously described methods (Watkins, H. (1994) Current Protocols in Human Genetics 7.1-7.2), two micrograms of total RNA obtained from EBV-tranformed lymphocytes were reverse transcribed using MMLV-RT (can be obtained from Gibco-BRL) and oligonucleotide 3930R in a 20 μl volume; the cDNA products were then amplified in a 50 μl PCR reaction using the outer primer pair 2761F and 3930R. The second round of PCR was performed with a final dilution of 1:1000 of the first round products, (e.g. using primers 2791F and 3900R, 3181F and 3391R, and 3651F and 3900R).

Direct Sequencing of PCR Products

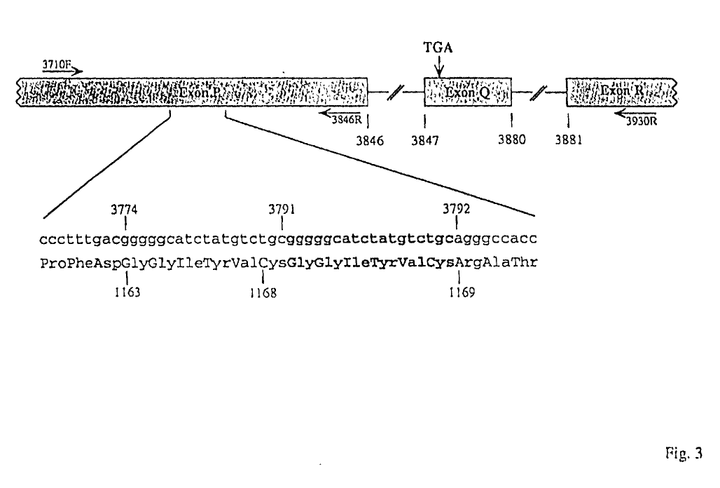

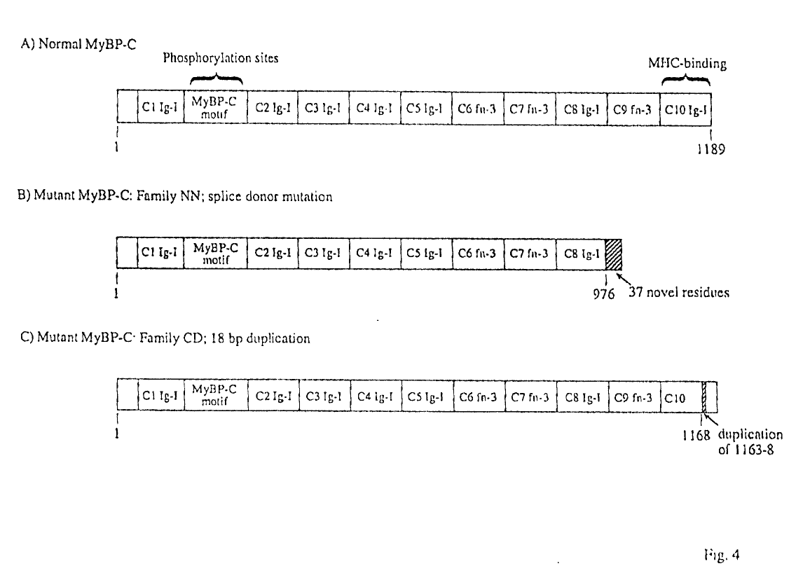

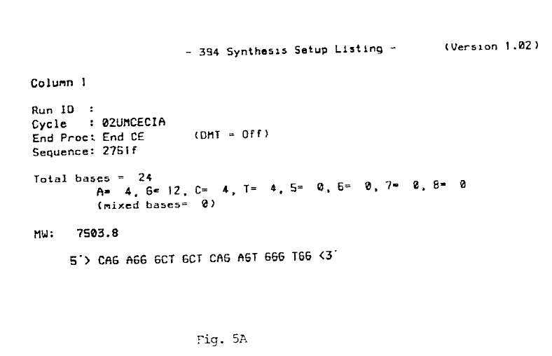

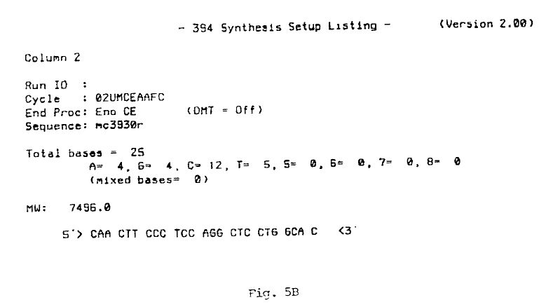

PCR amplified cardiac MyBP-C cDNA or genomic DNA fragments were sequenced using the cyclist™TaqDNA Sequencing Kit (Stragagene) according to instructions, except that the primer for sequencing was end-labeled with ³²Pγ ATP. The 5′ splice donor site mutation in Family NN was detected by sequencing the product amplified from genomic DNA by primers 3181F and 3391R with internal primer 3301F (FIG. 2). The duplication mutation in Family CD was defined by amplifying either cDNA or genomic DNA with primers 3710F and 3846R followed by sequencing of the longer allele with the same primers (FIG. 3). The sequence of the duplication was confirmed by sub cloning and sequencing of the longer allelle using the TA Cloning™ kit (Invitrogen).

Confirmation of Splice Donor Mutation by BstEII Digestion 15 μl of the PCR product amplified from genomic DNA by primers 3301F and 3391R was digested with 15U of BstEII, with addition of appropriate buffer to the PCR buffer, at 60° C. for 3 hours. Products were resolved on a resolved on a 3% Nusieve/1% agarose gel.

Linkage Analyses

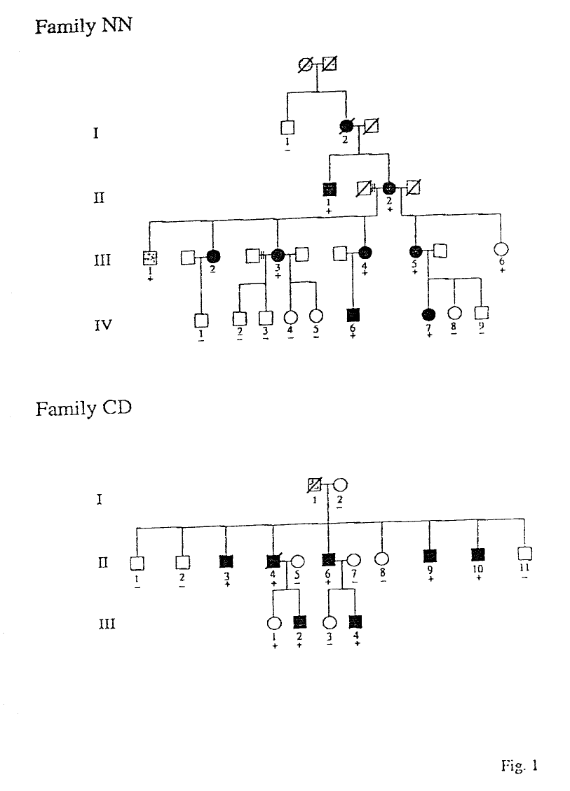

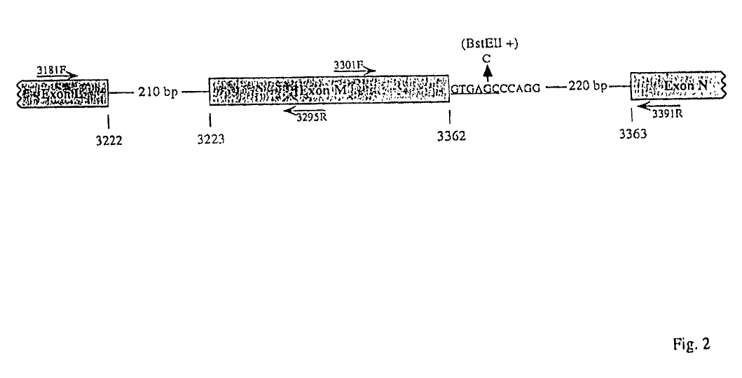

Linkage analyses were performed with affection status as indicated in FIG. 1 and disease penetrance of 90%. The allele frequencies for the G→C transversion and the duplication mutation in the cardiac MyBP-C gene were conservatively estimated at 0.01, based on their absence from 200 normal chromosomes. The theoretical maximum LOD scores under these conditions were 3.174 for Family NN and 3.32 for Family CD. Two point LOD scores were calculated using the computer program MLINK (Lathrop, G. M., et.al. (1984) Proc natl Acad Sci USA 81: 3443-3446).

### EXAMPLE 1

### Identification of Mutations in the Cardiac Myosin Binding Protein-C Gene

All members of two families with FHC (designated NN and CD. FIG. 1) were evaluated by physical examination, electrocardiogram, and 2-dimensional echocardiogram. In Family NN disease symptoms included exertional dyspnea and chest pain. One individual (IV-7) experienced syncope and another (Individual II-2) had a cerebral thromboembolism. There was no family history of sudden death. Seven individuals fulfilled standard diagnostic criteria for FHC (Watkins, H. et.al. (1995) N. Engl. J. Med. 332: 1058-1064). Individual III-5(age 50) lacked echocardiographic findings of cardiac hypertrophy but was also considered affected based on symptoms and nonspecific electrocardiographic abnormalities; in addition, she transmitted FHC to her daughter (Individual IV-7). Individual III-1 (age 35) had only non-specific electrocardiographic abnormalities and was considered to be of unknown disease status. Clinical studies in all other members of Family NN were normal.

Five adults in Family CD had typical signs and symptoms of FHC; one (Individual II-4) died suddenly at age 44 and post-mortem examination revealed marked (3 cm) ventricular septal hypertrophy. Two asymptomatic children without echocardiography evidence of cardiac hypertrophy also had findings consistent with disease: Individual III-2 (14 years) had an abnormal electrocardiogram: Individual III-4 (10 years) had systolic anterior motion of the mitral valve. The medical records of Individual IL-1 and I-2 were significant for cardiac disease; I-1 died during sleep at age 61 with a history of chest pain that had not been investigated; I-2 had evidence of prior myocardial infarction but not of FHC. All other members of Family CD had normal clinical findings.

Genetic analysis of both Family CD and NN excluded linkage to the three known FHC genes (Geisterfer-Lowrance, A. A. T., et.al. (1990) Cell 62: 999-1006. Thierfelder, L., et.al. (1994) Cell 77: 701-712, Watkins, H. et.al. (1995) N Engl. J. Med 332: 1058-1064). Linkage to CMH4 was assessed using flanking markers D11S905 and D11S905 which are separated by 17 cM (Carrier, L. et.al. (1993) Nature Genet 4: 311-313) showed no recombination in affected individuals genotyped in Family NN, and also identified a disease haplotype in Individual III-1 and clinically unaffected Individual III-6, Marker D11S987 was fully informative and concordant in Family CD except for a clinically unaffected 16 year old (III-1) who inherited the disease-associated allele. These genetic data and clinical findings suggested incomplete disease penetrance in both families, as has been seen in FHC families with mutations at this locus (Carrier, L. et.al. (1993) Nature Genet 4: 311-313) and other D11S905 loci (Watkins, H. et.al. (1995) N. Engl J Med. 332: 1058-1064).

Cardiac MyBP-C was mapped by FISH (Gautel, M., et al. (1995) EMBO J 14: 1952-1960) to the broad physical region containing the CMH4 locus (Carrier, L. et.al. (1993) Nature Genet 4: 311-313). To further refine the map location of cardiac MyBP-C we screened the CEPH B YAC library by PCR using two cDNA primers that span an intron (3301F and 3391R. Methodology). YAC clone 965-h-2 (on contig WC-476, (Whitehead Institute for Biomedical research/MIT Centre for Genome Research YAC database) contained cardiac MyBP-C. An adjacent YAC. 875-a-12, carries the polymorphism D11S1350, which is closely linked to the CMH4 locus (Carrier, L. et. al. (1993) Nature Genet. 4: 311-313, Gyapay, G. et.al. (1994) Nature Genet 7: 246-339). To determine if mutation of the cardiac MyBP-C gene caused FHC in Families NN and CD, lymphocyte RNA from two affected members of family was amplified by reverse-transcription and nested PCR. Oligonucleotides were synthesized based on the cDNA sequence (Gautel, M., et.al. (1995) EMBO J. 14: 1952-1960); the gene sequence and structure are not known.

Amplification of the 3′ region of cardiac MyBP-C cDNA (primers 2791F and 3900R, Methodology) in samples from affected individuals in Family NN yielded the expected 1110 bp product and also a shorter product. Amplification with internal primers 3181F and 3391R produced a cDNA that was 140 bp shorter than the wild-type but present in approximately equal amounts. To determine whether aberrant cDNA resulted from a deletion of abnormal splicing, the surrounding region was amplified from genomic DNA with primers 3181F and 3391R and sequenced directly. A 140 bp exon was identified, defined by residues 3223 to 3362 inclusive (denoted exon M. FIG. 2b); these same 140 residues were absent in the aberrant cDNA. The sequence of the 3′ splice acceptor site preceding exon M was identical in affected individuals and controls. However, a G→C transversion was identified at position 5 of the 5′ splice donor sequence GTGAGC in the following intron (FIG. 2). Samples from affected individuals contained the normal 210 bp product and also a 70 bp product resulting from skipping the 140 bp exon M.

The G→C transversion creates a new BstEII site, allowing independent confirmation of the mutation. Genomic DNA was amplified with primers 3301F and 3391R. In the mutant allele the gain of a BstEII site resulted in cleavage of normal 315 bp product into a 250 bp product and a 65 by product. All available clinically affected members of Family NN carried the mutation. Individuals III-1 and III-6 who both carried a disease-associated haplotype also had this mutation. The mutation was not present in the remaining unaffected family members nor in 200 chromosomes from unrelated, unaffected individuals. A LOD score of 2.48 at Θ≈0 was calculated by linkage analysis between the mutation and disease (Methodology).

Amplification of the 3′ region of the cardiac MyBP-C cDNA (primers 3651F and 3900R, Methodology) in samples from affected members of Family CD yielded the expected 250 bp product but also an abnormal longer transcript (a 268 bp product resulting from the 18 bp duplication). Amplification of genomic DNA with the same primers similarly revealed an additional longer product. Sequencing of the longer cDNA product identified an 18 base-pair tandem duplication of residues 3774-3791; sequencing of the genomic product confirmed in duplication, which occurs in the penultimate exon of the coding sequence (denoted exon P. FIG. 3). Amplification of genomic DNA with primers within exon P (3710F and 3846R) demonstrated the duplication in samples from all affected members of Family CD and also the presumed non-penetrant 16 year old, III-1. In individuals heterozygous for the mutant allele the duplication results in a 155 bp product in addition to the normal 137 bp product Six clinically affected individuals and one clinically unaffected individual (III-1) carry the mutation. The mutation was not present in the remaining unaffected family members nor in 200 chromosomes from unrelated, unaffected individuals. A LOD score of 2.32 at Θ=0 was calculated by linkage analysis between the mutation and disease (Methodology).

Both mutations in the cardiac MyBP-C gene cause FHC because they segregate with disease (although with incomplete penetrance), are not present in controls, and result in aberrant cDNAs that are predicted to encode significantly altered MyBP-C polypeptides. The G₅ residue is a highly conserved nucleotide in the splice donor consensus sequence (Shapiro, M. B., et al. (1987) Nucl. Acids Res. 15: 7155-7174); the G→C transversion found in Family NN appears to completely inactivate this donor site. The resultant skipping of the exon in lymphocyte cDNA is an expected consequence in the absence of an alternative splice donor site in the intron (Green, M. R. (1986) Ann. Rev. Genet. 671-708, Robberson, B. L., et.al. (1990) Mol. Cell. Biol. 10: 84-94). Although heart tissue is unavailable from affected individuals in this family, similar consequences of the donor splice site mutation in the myocardium are expected. Skipping the 140 bp exon M produces a frame-shift; the aberrant cDNA encodes 976 normal cardiac MyBP-C residues, then 37 novel amino acids, followed by premature termination of translation (successive TAG and TGA codons, FIG. 4). Two hundred and thirteen amino acids are deleted from the conserved carboxy-terminus (56%) identity with chicken fast skeletal MyBP-C (Gautel, M., et.al. (1995) EMBO J. 14: 1952-1960). The mutations found in Family CD also affects the conserved carboxy-terminus with the tandem duplication of six amino acid residues: GlyGlyIleTyrValCys (residues 1163-1168, FIG. 4). This duplication involves a consensus sequence (GlyXTyrXCys) within the immunoglobulin C2 domain (Williams, A. F., et.al. (1988) A. Rev Immun. 6: 381-405) and is predicted to disrupt one of seven β sheets that form the conical 3-dimensional barrel structure (Okagaki, T., et.al. (1993) J Cell Biol. 123: 619-626).

Cardiac MyBP-C is the predominant myosin binding protein in the heart and is not expressed in other tissues (Gautel, M., et.al. (1995) EMBO J. 14: 1952-1960); mutations would therefore be expected to produce the cardiac-specific phenotype of FHC. Cardiac MyBP-C is thought to participate in thick filament assembly by binding myosin heavy chain titin (Schultheiis, T., et.al. (1990)J. Cell Biol 110: 1159-1172). In addition, the protein has regulatory functions: interactions with F-actin and the myosin head modulate myosin ATPase (Moos, C., et.al. (1980) Biochim Biophys. Acta 632: 141-149); reversible phosphorylation of cardiac MyBP-C by cAMP-dependent protein kinase (Gautel, M., et al. (1995) EMBO J 14: 1952-1960) and caladium calmodulin-dependent protein kinase II (Schlender, K., et.al. (1991) J. Biol Chem. 266: 2811-2817) also participates in adrenergic regulation of cardiac contraction. Both the mutated MyBP-C polypeptides described here should contain phosphorylation sites (Gautel, M., et.al. (1995) EMBO J. 14: 1952-1960) and regions that interact with titin (Furst, D. O., et.al. (1992) J Cell Sci. 102: 769-778). However, the high-affinity myosin binding domain is confined to the highly conserved carboxy-terminal (C10) immunoglobulin-like repeat (Okagaki, T., et.al. (1993) J Cell biol 123: 619-626) which is absent in the truncated polypeptide due to the splice donor mutation and is interrupted by the duplication (FIG. 4). Both mutant alleles might therefore be expected to encode an MyBP-C capable of associating with some sarcomeric proteins, but defective in binding the myosin heavy chain rod.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for detecting the presence or absence of a mutation associated with hypertropic cardiomyopathy, comprising:
providing DNA which encodes a cardiac myosin binding protein; and
detecting the presence or absence of a mutation in the DNA which is associated with hypertrophic cardiomyopathy.

2. The method of claim 1 further comprising amplifying the DNA to form an amplified product and detecting the presence or absence of a mutation in the amplified product which is associated with hypertrophic cardiomyopathy.

3. The method of claim 1 wherein the cardiac myosin binding protein is cardiac myosin binding protein-C.

4. The method of claim 1 wherein the hypertrophic cardiomyopathy is familial hypertrophic cardiomyopathy.

5. The method of claim 1 wherein the hypertrophic cardiomyopathy is sporadic hypertrophic cardiomyopathy.

6. The method of claim 1 wherein the mutation is the cardiac myosin binding domain.

7. The method of claim 1 wherein the mutation is a splie site mutation.

8. The method of claim 1 wherein the mutation is a duplication mutation.

9. The method of claim 1 wherein the DNA is cDNA reversed transcribed from RNA.

10. The method of claim 9 wherein the RNA is obtained from nucleated blood cells.

11. The method of claim 1 wherein the presence or absence of the mutation associated with hypertrophic cardiomyopathy is detected by contacting the DNA with an RNA probe completely hybridizable to DNA which encodes a normal cardiac myosin binding protein to form a hybrid double strand having an RNA and DNA strand, the hybrid double strand having an unhybridized portion of the RNA strand at any portion corresponding to a hypertrophic cardiomyopathy-associated mutation in the DNA strand; and
detecting the presence or absence of an unhybridized portion of the RNA strand as an indication of the presence or absence of a hypertrophic cardiomyopathy-associated mutation in the corresponding portion of the DNA strand.

12. The method of claim 2 wherein the DNA which encodes a cardiac myosin binding protein is amplified using a polymerase chain reaction.

13. The method of claim 12 wherein the polymerase chain reaction is a nested polymerase chain reaction.

14. A method for diagnosing familial hypertrophic cardiomyopathy in a subject, comprising:
obtaining a sample of DNA which encodes a cardiac myosin binding protein from a subject being tested for familial hypertrophic cardiomyopathy;
diagnosing the subject for familial hypertrophic cardiomyopathy by detecting the presence or absence of a mutation in the cardiac myosin binding protein which causes familial hypertrophic cardiomyopathy as an indication of the disease.

15. A method for detecting the presence or absence of a mutation associated with hypertrophic cardiomyopathy, comprising:
providing DNA which encodes a cardiac myosin binding protein; and
detecting the presence or absence of a mutation in the DNA which is associated with hypertrophic cardiomyopathy.

16. A non-invasive method for diagnosing hypertrophic cardiomyopathy, comprising:
obtaining a blood sample from a subject being tested for hypertrophic cardiomyopathy;
isolating cardiac myosin binding protein RNA from the blood sample; and
diagnosing the subject for hypertrophic cardiomyopathy by detecting the presence or absence of a mutation in the RNA which is associated with hypertrophic cardiomyopathy as an indication of the disease.

17. The method of claim 16 wherein the presence or absence of a mutation associated with hypertrophic cardiomyopathy in the RNA is detected by preparing cardiac myosin binding protein cDNA from the RNA to form cardiac myosin binding protein DNA and detecting mutations in the DNA as being indicative of mutations in the RNA.

18. The method of claim 16 further comprising amplifying the cardiac myosin binding protein DNA prior to detecting a mutation in the DNA which is associated with hypertrophic cardiomyopathy.

19. The method of claim 16 wherein the hypertrophic cardiomyopathy is familial hypertrophic cardiomyopathy.

20. The method of claim 16 wherein the hypertrophic cardiomyopathy is sporadic hypertrophic cardiomyopathy.

21. The method of claim 16 further comprising evaluating the subject for clinical symptoms associated with hypertrophic cardiomyopathy.

22. A kit useful for diagnosing hypertrophic cardiomyopathy, comprising:
a first container holding an RNA probe completely hybridizable to DNA which encodes a cardiac myosin binding protein; and
a second container holding primers useful for amplifying the DNA which encodes a cardiac myosin binding protein.

23. A kit of claim 22 further comprising a third container holding an agent for digesting unhybridized RNA.

24. The kit of claim 22 further comprising instructions for using the components of the kit to detect the presence or absence of mutations in amplified DNA which encodes a cardiac myosin binding protein.

25. The kit of claim 22 wherein the DNA encodes cardiac myosin binding protein-C.

26. A non-human animal embryo comprising DNA which encodes a cardiac myosin binding protein, the DNA having at least one hypertrophic cardiomyopathy-causing mutation in its nucleotide sequence.

27. A non-human animal comprising DNA which encodes a cardiac myosin binding protein, the DNA having at least one hypertrophic cardiomyopathy-causing mutation in its nucleotide sequence.

28. A method for screening an agent for its ability to treat hypertrophic cardiomyopathy in a subject, comprising:
providing a non-human animal comprising DNA which encodes a cardiac myosin binding protein, the DNA having at least one hypertrophic cardiomyopathy-causing mutation in its nucleotide sequence;
administering an agent being tested for its ability to treat hypertrophic cardiomyopathy in a subject to the non-human animal; and
determining the effect of the agent on the hypertrophic cardiomyopathy in the non-human animal.

29. A method for treating hypertrophic cardiomyopathy in a subject, comprising:
providing DNA which encodes a normal cardiac myosin binding protein; and
administering the DNA to a subject having hypertrophic cardiomyopathy such that the hypertrophic cardiomyopathy is treated.
